(19) United States
(12) Patent Application Publication
(10) Pub. No.: US 2025/0000874 A1
(43) Pub. Date: Jan. 2, 2025

(54) BACTERIAL METABOLITES AND THEIR USE IN REDUCING INFLAMMATION

(71) Applicant: LOYOLA UNIVERSITY OF CHICAGO, Chicago, IL (US)

(72) Inventors: Liang Qiao, Chicago, IL (US); Juan Reyes, Chicago, IL (US); Ping Bu, Chicago, IL (US); Ryan Compas, Chicago, IL (US); An Nguyen, Chicago, IL (US); Frank Gambino, JR., Chicago, IL (US); Rim Noufal, Chicago, IL (US); Drew Landrowski, Chicago, IL (US); Reynolds Boone, Chicago, IL (US)

(21) Appl. No.: 18/667,386

(22) Filed: May 17, 2024

Related U.S. Application Data
(60) Provisional application No. 63/467,646, filed on May 19, 2023.

(51) Int. Cl. A61K 31/573; A61K 31/198; A61K 31/566; A61K 35/74; A61P 27/02; A61P 29/00

(52) U.S. Cl. CPC A61K 31/573; A61K 31/198; A61K 31/566; A61K 35/74; A61P 27/02; A61P 29/00

(57) ABSTRACT

The disclosure provides a composition comprising (a) one or more isolated bacterial metabolites selected from prednicarbate, N-palmitoyl-D-threonine and 3,3-difluoro-5-alpha-androstan-17-beta-yl-acetate, and (b) a pharmaceutically acceptable carrier, and use of such composition for reducing inflammation and/or treating an inflammatory disorder in a subject in need thereof.

### BACKGROUND

The human body contains a diverse ecosystem of microbes. Commensal bacteria have numerous mutually beneficial relationships with their host. Commensal bacteria not only assist in digestion and gut homeostasis, but they are also critical for development and function of the immune system. As such, hosts and microbes alike have evolved mechanisms to foster symbiosis. Gut barrier function, toll-like receptor signaling, and metabolites are all examples of how hosts interact with microbes to promote this mutually beneficial relationship. Metabolites from commensal bacteria promote the generation of regulatory T cells, capable of ameliorating inflammation. There remains a need in the art to identify metabolites from commensal bacteria that demonstrate an anti-inflammatory effect.

### SUMMARY

In one aspect, described herein is a composition comprising (a) one or more isolated bacterial metabolites selected from prednicarbate, N-palmitoyl-D-threonine and 3,3-difluoro-5-alpha-androstan-17-beta-yl-acetate, and (b) a pharmaceutically acceptable carrier.

In another aspect, described herein is a method of reducing proinflammatory cytokine expression in a subject in need thereof, comprising administering the composition described herein to the subject in an amount effective to reduce proinflammatory cytokine expression in the subject.

In another aspect, described herein is a method of reducing inflammation in a subject in need thereof, comprising administering the composition described herein to the subject in an amount effective to reduce inflammation.

In another aspect, described herein is a method of treating an inflammatory disorder in a subject in need thereof, comprising administering the composition described herein to the subject in an amount effective to treat the disorder.

### BRIEF DESCRIPTION OF THE FIGURES

FIG. 1A-1D. Bacterial Supernatant <10 kD fraction inhibits macrophage activation in both mouse and human macrophages. Mouse BMDM, (FIG. 1A, FIG. 1B) or PMA differentiated human THP-1 macrophages (FIG. 1C, FIG. 1D) were pretreated with bacterial supernatant <10 kD fraction overnight followed by LPS stimulation for 4 hr. Then, secretions of TNFα and IL-6 were analyzed by ELISA. **, P<0.01.

FIG. 2A-2D. Bacterial supernatant <10 kD fraction inhibits NLRP3 inflammasome activation. Mouse BMDMs (FIG. 2A, FIG. 2B, FIG. 2C) or PMA differentiated human THP-1 macrophages (FIG. 2D) were primed with 50 ng LPS overnight and then treated with bacterial supernatant samples for 6 hrs. Cells were then stimulated with 3 mM ATP for 1 hr or 200 ug MSU overnight. Secretions of IL-1B or TNFα were analyzed by ELISA. *, P<0.05, **, P<0.01.

FIG. 3A-3B. Bacterial metabolite(s) can pass through intestinal epithelial cells. Caco-2 cells were grown in upper chambers of transwell. THP-1 macrophages were plated in the lower wells and plated in the lower wells. (FIG. 3A) Whole supernatant, >10 kD, <10 kD fractions of LF or TS media were added in upper chambers and incubated overnight, followed by LPS stimulation in upper or lower wells for 6 hr. (FIG. 3B) THP-1 macrophages were primed with 50 ng LPS overnight. Then treatments were added into upper chambers for 6 hr followed by stimulating macrophages in the lower wells overnight with 200 ug MSU. Cytokine secretions were analyzed by ELISA. *, P<0.05, ***, P<0.001.

FIG. 4A-4B. Bacterial Supernatant <10 kD fraction inhibits mouse peritonitis in vivo. 6-week old B6 mice were i.p. injected with <10 kD fraction from LF, TS medium, or PBS (500 μL/dose) at day 0 and day 1. At day 2, mice were given LPS (10 μg/mouse) or PBS via i.p. Six hours later, mice were sacrificed and the peritoneal cavities were washed with 5 mL PBS. (FIG. 4A) The recruited neutrophils were quantified by flow cytometry using the neutrophil markers Ly6G and data were analyzed by FlowJo Software. (FIG. 4B) Neutrophil numbers are shown as mean±s.d., **p<0.01, n=3 per group.

FIG. 5A-C. Bacteria metabolites (MQ, JE, and NC) reduce expression of pro-inflammatory cytokines. Mouse macrophages cells, (FIG. 5A, FIG. 5B) Human Corneal Epithelial Cells (FIG. 5C) or THP-1 macrophages (FIG. 5D) were pre-treated with either 10 μM (3.5 μg/mL), 50 μM (17 μg/mL) of JE, 10UM (4.9 μg/mL), 50 μM (24 μg/mL) of MQ or combination of both for 16 hours, followed by 200 ng/mL of LPS to Mouse macrophages/THP1 cells, and 100 ng/ml of Flagellum to HCECs for 3 hours. The supernatants were collected and TNFα, IL-6 concentration was measured by ELISA. *, P<0.05, **. P<0.01, ***, P<0.001.

FIG. 6. Bacteria metabolites (MQ, JE, and NC) reduce IL-6 expression. THP-1 macrophages were pre-treated with either 10 μM (3.5 μg/mL) of JE, 10 μM (4.9 μg/mL) of MQ or 50UM of NC or different combinations of all three for 16 hours, followed by 200 ng/ml of LPS for three hours. Supernatants were collected and IL-6 concentration was measured by ELISA. **. P<0.01.

FIG. 7A-7B. Bacteria metabolites (MQ and JE) reduce IL-1β expression. Murine macrophages, (FIG. 7A) or THP-1 macrophages (FIG. 7B) were pre-treated with either 10 μM (3.5 μg/mL), 50 μM (17 μg/mL) of JE, 10 μM (4.9 μg/mL), 50 μM (24 μg/mL) of MQ or combination of both for 16 hours. Macrophages were primed with 200 ng/ml of LPS for three hours, followed by inflammasome activation with 5 mM ATP for one hour. Supernatants were collected, IL-1B was measured by ELISA. ***, P<0.001

FIG. 8A-8B. The assessment of the permeability of the differentiated CACO-2 epithelium cells after the treatment of JE, MQ, and NC in trans-well system. (FIG. 8A) CACO-2 cells were seeded in the upper well, and B6 Macrophages were placed into the lower well. 50UM of JE, MQ, NC, or combination of all three was added to CACO-2 cells in the upper well. Samples and controls were incubated overnight at 37° C., 5% CO₂, followed by the addition of 200 ng/ml LPS to the upper and lower wells for 3 hours (Left: lower chambers, right: upper chambers). Cell culture supernatants were collected and an ELISA was used to measure TNFα or IL-6 concentrations. (FIG. 8B) FitC-dextrans (4 KDa) were added into the upper chamber simultaneously as the sample tests were added. A high concentration of hydrochloric acid (HCl) as a control was added to the upper chamber to disrupt the tight junctions. FitC-dextran collected from the bottom chamber was measured by a fluorescence spectrometer (excitation, 490 nm; emission, 520 nm). Data shown are representative mean±s.d. (***, p<0.001, **, p<0.01).

FIG. 9A-9C. FACS representation of mouse peritonitis model. Histograms were made from FACS gating of cells collected from the peritoneal cavity. (FIG. 9A) Mice were pre-treated for two consecutive days with compound JE and then received LPS. (FIG. 9B) Mice received only LPS. (FIG. 9C) Another group received PBS injected for two consecutive days to serve as a negative control. Mice were euthanized four hours after the addition of LPS and cells were then extracted from the peritoneal cavity. The collected cells were blocked with Fc-blocking antibody and then stained with neutrophil marker PE-anti mouse Ly6G. Histograms of FACS staining for Ly6G are shown above showing the percentage of PE+ cells for each group.

FIG. 10. Toxicity Assay shows high viability for 50 μM JE. IC-21 murine peritoneal macrophages were seeded at a density of 2.0×10⁴ per well in a 96-well plate. Differing concentrations of compound JE ranging from 0-800 M were diluted in DMSO and added to the appropriate wells. Cells were incubated at 37° C. for 48 hours. After the incubation period, Cell Counting Kit-8 (Sigma-Aldrich) was used for quantification of viable cells. CCK-8 solution was added to each well and the plate was incubated at 37° C. for two hours. Levels of water-soluble tetrazolium salt reduced to formazan were then measured at a wavelength of 450 nm. Cell viability was found as a percentage by dividing each absorbance value by the negative control.

FIG. 11. Probiotic bacteria metabolite JE inhibits TLR5 agonist-induced IL-6 in HCECs. HCECs were pretreated with 50 M JE overnight and then stimulated with 0.1 μg/ml flagellin for the indicated time. The effect of TLR5 agonist (flagellin) on IL-6 secretion were measured in cell culture supernatants by ELISA. *, P<0.05.

FIG. 12. Corneal Florescein Staining with JE-treated Experimental Dry Eye. Mean corneal staining score with JE/control topical treatment (4 times/day) starting 3 weeks after housing in ICES. Group 1: Normal control (non DED); group 2: Dry Eye treated with JE (JE+DED); Group 3: Dry Eye treated with vehicle (control DED). P value: *: <0.05. One of two experiments is shown.

FIG. 13. The Effect of JE on TNF-α Production by Raw 264.7 Cells Upon Stimulation with TLR Agonists. Macrophages were incubated overnight with JE at a concentration of 50 μM at t 37° C., 5% CO₂. The following day, the macrophages were stimulated with 10 ng/ml of LPS, 1 μg/ml of FLG-St, 3 μg/ml of PAM-3csk, 1 μM of ODN and 20 μg/ml of Poly: IC for 3 hours. Supernatants were collected, and TNF-α was measured by ELISA. The data are presented as mean±SEM (n=6). *p<0.05; **p<0.01; ***p<0.001. One of three experiments is shown.

FIG. 14. The Effect of JE on TNF-α Production by Raw 264.7 Cells Upon Stimulation with TLR Agonists. Macrophages were incubated overnight with JE at a concentration of 50 μM at 37° C., 5% CO₂. The following day, the macrophages were stimulated with 1 μg/ml of FSL-1 2 μg/ml of Imiquimod for 3 hours. Supernatants were collected and TNF-α was measured by ELISA. Data are presented as mean±SEM (n=6). *p<0.05; **p<0.01; ***p <0.001. One of three experiments is shown.

FIG. 15. The Effect of JE on IL-6 Production by Raw 264.7 Cells Upon Stimulation with TLR Agonists. Macrophages were incubated overnight with JE at a concentration of 50 μM at t 37° C., 5% CO₂. The following day, the macrophages were stimulated with TLR agonists at the same concentrations that I used for TNF-α induction. Supernatants were collected, and IL-6 was measured by ELISA. The data are presented as mean±SEM (n=6). *p<0.05; **p<0.01; ***p<0.001. One of three experiments is shown.

FIG. 16. The Effect of JE on TNF-α Secretion by BMDMs in Response to Stimulation by Different Concentrations of LPS. BMDMs were isolated and cultured, as mentioned before. Macrophages were incubated overnight with JE at a concentration of 50 μM at 37° C., 5% CO2. The next day, they were stimulated with two-fold dilutions of LPS at a starting concentration of 5 ng/ml for 3 hours. Supernatants were collected, and TNF-α was measured using ELISA. Data are presented as mean±SEM (n=6). *p<0.05; **p<0.01; ***p<0.001. One of two experiments is shown.

FIG. 17. The Effect of JE on IL-6 Secretion by BMDMs in Response to Stimulation by Different Concentrations of LPS. BMDMs were isolated and cultured as described above. Macrophages were incubated overnight with JE at a concentration of 50 μM at 37° C., 5% CO₂. The next day, they were stimulated with two-fold dilutions of LPS at a starting concentration of 5 ng/ml for 3 hours. Supernatants were collected, and IL-6 was measured using ELISA. Data are presented as mean±SEM (n=6). *p<0.05; **p<0.01; ***p<0.001. One of two experiments is shown.

FIG. 18. The Effect of JE on TNF-α Secretion by BMDMs in Response to Stimulation by TLR Agonists. BMDMs were isolated and cultured as described above. Macrophages were incubated overnight with JE at a concentration of 50 μM at 37° C., 5% CO₂. The following day, they were stimulated with 1 μg/ml of FLG-ST, 1 mM and 0.5 mM of ODN, 50 μg/ml of Poly: IC, 3 μg/ml of Imiquimod, 3 μg/ml and 1.5 μg/ml of PAM-3SCk, 1 μg/ml of FSL-1 for 3 hours. Supernatants were collected, and TNF-α was measured using ELISA. Data are presented as mean±SEM (n=6). *p<0.05; **p<0.01; ***p<0.001. One of three experiments is shown.

FIG. 19. The Effect of JE on IL-1β Production by BMDMs Upon NLRP3 Activation. Macrophages were pretreated with the metabolite JE at a concentration of 50 μM overnight at 37° C., 5% CO₂. The following day, macrophages were primed with LPS at 500 ng/ml for 4 hours and then activated by ATP for 1 hour at 37° C., 5% CO₂. Another approach is to prime the cells with 10 ng/ml LPS for 4 hours, then pretreat with JE overnight. The following day, macrophages were activated with 5 mM ATP for an hour at 37° C., 5% CO₂. Supernatants were collected, and IL-β was measured using ELISA. Data are presented as mean±SEM (n=6). **p<0.01. One of three experiments is shown.

FIG. 20. The Effect of JE on IL-1β Production by BMDMs Upon NLRP3 Activation. Macrophages were pretreated with the metabolite JE at a concentration of 50 μM overnight at 37° C., 5% CO2. The following day, macrophages were primed with LPS at 500 ng/ml for 4 hours and then activated by ATP for 1 hour at 37° C., 5% CO2. Another approach is to prime the cells with 10 ng/ml LPS for 4 hours, then pretreat with JE overnight. The following day, macrophages were activated with 5 mM ATP for an hour at 37° C., 5% CO2. Supernatants were collected, and IL-β was measured using ELISA. Data are presented as mean±SEM (n=6). **p<0.01. One of three experiments is shown.

FIG. 21. The Impact of JE on Caspase-1 Activation After Priming the Cells with LPS. BMDMs were cultured and seeded as described before. Macrophages were first primed with LPS for 4 hours at 37° C. with 5% CO₂. After that, they were treated with the metabolite JE for 1 (LPS1ATP), 2 (LPS2ATP), and 3 (LPS3ATP) hours. Then, the cells were exposed to media containing 5 mM ATP for 1 hour at 37° C. with 5% CO₂. Finally, the supernatants were collected and the level of IL-1β was measured using ELISA. Data are presented as mean±SEM (n=6). *p<0.05; ***p<0.001. One of two experiments is shown.

FIG. 22. The Effect of JE on IL-1β Production by THP-1 Macrophages. THP-1 macrophages were pretreated with JE overnight at 37° C., 5% CO₂. The next day, they were primed with LPS at 500 ng/ml for 4 hours then activated with 6.5 μM of nigericin for 30 minutes at 37 C, 5% CO2. Supernatants were collected and IL-1β production with ELISA. Data are presented as mean±SEM (n=6). *p<0.05. One of two experiments is shown.

FIG. 23. The Effect of JE on IL-1β Production by THP-1 Macrophages: JE was used as a pretreatment when THP-1 macrophages were pretreated with JE overnight at 37° C., 5% CO2. The next day, they were primed with LPS at 500 ng/ml for 4 hours, then activated with 5 mM of ATP for 30 minutes at 37° C., 5% CO2. In the case when JE was used as a treatment, cells were primed with 10 ng/ml of LPS for 4 hours, then JE was added overnight at 37° C., 5% CO2. The following day, macrophages were activated with 5 mM of ATP for an hour. Supernatants were collected and IL-1β production with ELISA. Data are presented as mean±SEM (n=6). **p<0.01. One of two experiments is shown.

FIG. 24. Priming with LPS is Sufficient to Trigger IL-1β Production by THP-1 Macrophages. Macrophages were stimulated with 500 ng/ml and 1 μg/ml of LPS for 4 hours or 50 ng/ml and 100 ng/overnight at 37° C., 5% CO2. Supernatants were collected and IL-1β production was measured by ELISA. Data are presented as mean±SEM (n=6). **p<0.01. One of three experiments is shown.

FIG. 25. Pro-Inflammatory IL-6 Cytokine Expression in Corneal Tissues Isolated from Control, Vehicle Control, and Treatment Groups. Corneal samples from the three groups were isolated, stabilized in RNA later, and stored at-80. Corneal samples were homogenized, and RNA was purified using RNAeasy mini-kit. A total of 0.5 μg-1 μg RNA was yielded. RT-qPCR was performed. Values were normalized to GAPDH control, and fold induction was measured as 2-ΔΔCT. One of two experiments is shown.

FIG. 26. MQ Reduces IL-6 Response to Toll 3 and Toll 5 Agonist. HCECs were cultured at a uniform density and allowed to adhere. Cells were then incubated overnight in MQ at a concentration of 60 μM at 37° C. The following day, the HCECs were stimulated with FLA at a concentration of 100 ng/ml or P (I:C) at 500 μg/mL for four hours. Supernatants were collected and IL-6 concentrations were determined via ELISA.

FIG. 27A and FIG. 27B. Metabolite NC on TNFα release in response to LPS in human and murine macrophages in vitro. Macrophages were plated at a uniform density of 0.2×10⁶ cells/mL in 24 well plates. These cells (FIG. 27A) were allowed to adhere, or, in the case of THP-1 cells FIG. 27B), differentiate using PMA. They were then treated for 18 hours with 50 μM of metabolite NC before being stimulated with 0.1 ug of LPS-EK. Supernatant was collected between 4-6 hours after addition of LPS and debris was removed via centrifugation at 1250 rpm for 5 minutes. TNFα was quantified in the supernatant using sandwich ELISA with standards of known concentrations. Cells treated with NC prior to stimulation released significantly less (p=0.000045 and p<0.00001, respectively, student's t-test) TNFα in response to LPS stimulation. Likewise, NC does not increase TNFα release in unstimulated cells.

FIG. 28A and FIG. 28B. Metabolite NC on TNFα release in macrophages in response to various TLR agonists. Raw 264.7 cells (FIG. 28A) and THP-1 FIG. 28B) cells were plated at a uniform density of 0.2×10⁶ cells per mL and stimulated with various TLR agonists: Pam3CSK4, Poly I:C, LPS, Fla, FSL-1, imiquimod (ImiQ), and ODN-2006. NC reduced TNFα release in response to TLR3 (p<0.0001 via student's t-test for both Raw 264.7 cells and THP-1 cells) and TLR4 agonists in both cell lines. NC did not significantly reduce TNFα release in response to other TLR agonists.

FIG. 29. HCEC responsiveness to various TLR agonists. HCECs were stimulated with agonists for TLR 1/2 (Pam3CSK4), TLR3 (Poly I:C), TLR4 (LPS), TLR5 (Flagellin), TLR6 (FSL-1), TLR7 (Imiquimod), and TLR9 (ODN2006). Supernatant was collected at 6-8 hours and debris was removed via centrifugation at 1250 rpm for 5 minutes. IL-6 in the supernatant was then quantified using a sandwich ELISA for human IL-6 with a standard of known IL-6 concentration. HCECs only respond to stimulation by TLR3 and TLR5.

FIG. 30. NC on IL-6 Release in HCECs. Human corneal epithelial cells were plated at a uniform density of 0.2×10⁶ cells/mL in 24 well plates. These cells were allowed to adhere for 6-8 hours. They were then treated 18 hours with 50tM of metabolite NC before being stimulated with 1 tg/mL of PA-Flagellin or 50 tg/mL Poly I:C. Supernatant was collected between 6-8 hours after addition of TLR agonist and centrifuged at 1250 rpms for 5 minutes to remove any debris. IL-6 in the supernatant was then quantified using a sandwich ELISA for human IL-6 with a standard of known IL-6 concentration. NC does not reduce IL-6 release in response to Poly I:C or Flagellin in HCECs.

FIG. 31. Compound NC on phospho-NFκB (ser536) levels in stimulated macrophages. Raw 264.7 macrophages were incubated with 50 μM of NC for 18 hours prior to stimulation with LPS or Poly I:C. Whole cell lysates were collected after 2 hours and the level of phosphorylated NFκB (Phosphoserine 536) was quantified for endpoint absorbance at λ=450 nM and normalized to absorbance of untreated, unstimulated cells. Mean and p values were computed using R base and dplyr packages using normalized values. These findings indicate that NC treatment significantly decreases active NFκB levels in cells treated with Poly I:C (p=0.0013, student's t-test of absorbance values) and LPS (p=0.0338, student's t-test of absorbance values). This indicates that NC acts on NFκB through TRIF to decrease TNFα transcription.

FIG. 32. NC Reduces TNFα Response to Toll 3 Agonist. Macrophages were cultured at a uniform density and allowed to adhere (in the case of BMDMs) or differentiate with PMA (in the case of THP-1s). macrophages were then incubated overnight in NC at a concentration of 50 uM at 37° C. The following day, the BMDMs were stimulated with P(I:C) at 50 ug/mL, 100 ug/mL, or 200 ug/mL for four hours and the THP-1s were stimulated at 200 mg/mL or 400 mg/mL. Supernatants were collected and TNFα concentrations were determined via ELISA.

FIG. 33. TNFα production from Bone Marrow Derived Macrophage (BMDM) stimulation. BMDMs were cultured in DMEM supplemented with Macrophage Colony Stimulating Factor (M-CSF) at 10 ng/ml. The macrophages were then incubated overnight in NC at a concentration of 50 uM at 37° C. The following day they were stimulated with LPS at 1 ng/ml, 10 ng/ml for four hours. Supernatants were collected and TNFα concentrations were determined via ELISA.

FIG. 34. TNFα production from THP-1 stimulation. Thp-1s plated at uniform density and incubated in PMA to differentiate and treated with NC overnight. The following day, cells were then treated with FSL at a concentration of 20 ng/ml and 50 ng/ml. After 4 hours, supernatant was collected and the concentration of TNFα was determined via ELISA.

### DETAILED DESCRIPTION

This disclosure relates to compositions and therapeutic methods for inhibiting inflammatory mechanisms in tissues and/or treating and preventing inflammatory and/or autoimmune disorders. The present disclosure is based on the discovery that one or more isolated bacterial metabolites selected from prednicarbate, N-palmitoyl-D-threonine and 3,3-difluoro-5-alpha-androstan-17-beta-yl-acetate reduce inflammatory molecules in tissues of a subject.

Chemical structure of Prednicarbate (C₂₇H₃₆O₈), (“MQ”):

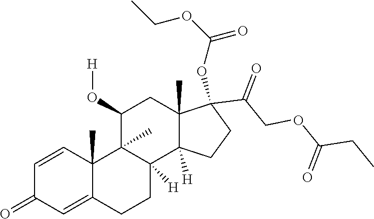

Chemical structure of N-Palmitoyl-D-Threonine (2˜{R},3˜{S})-2-(hexadecanoylamino)-3-hydroxybutanoic acid), (“NC”):

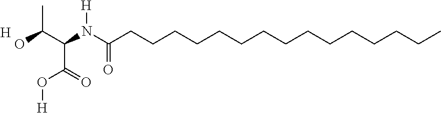

Chemical Structure of 3,3-Difluoro-5-alpha-androstan-17-beta-yl acetate, (“JE”):

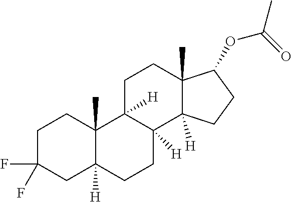

The compositions can contain prednicarbate (MQ), N-palmitoyl-D-threonine (NC), and/or 3,3-difluoro-5-alpha-androstan-17-beta-yl-acetate (JE) in any suitable concentration. Alternatively, or additionally, when the compositions contain two or more of MQ, NC, and JE, any pair of compounds (e.g., MQ/NC, MQ/JE, NC/JE) can be present in a relative amount expressed as a weight ratio or a molar ratio between the two compounds. Suitable ratios can be 100:1 to 1:100, 50:1 to 1:50, 20:1 or 1:20, 10:1 to 1:10, 5:1 to 1:5, 2:1 or 1:2, for example at least 100:1, 80:1, 60:1, 50:1, 35:1, 20:1, 10:1, 8:1, 6:1, 5:1, 3.5:1, 2:1, 1.5:1, 1.2:1, 1:1, 1:1.2, 1:1.5, 1:2, 1:3.5, 1:5, 1:6, 1:8, 1:10, 1:20, 1:35, or 1:50, and/or up to 50:1, 35:1, 20:1, 10:1, 8:1, 6:1, 5:1, 3.5:1, 2:1, 1.5:1, 1.2:1, 1:1, 1:1.2, 1:1.5, 1:2, 1:3.5, 1:5, 1:6, 1:8, 1:10, 1:20, 1:35, 1:50, 1:60, 1:80, or 1:100. The foregoing ratios and ranges can apply to a single composition containing the MQ, NC, and/or JE compounds. Alternatively, or additionally, the foregoing ratios can apply to a plurality of compositions collectively containing the MQ, NC, and/or JE compounds, such that the ratios reflect the total amount of the compounds administered to a patient in a single dose or over time (e.g., administering 2 units of MQ from a first composition and administering 1 unit of NC from a second composition for a cumulative or net MQ: NC ratio of 2:1). Independent or different ratios or ranges can be selected for each of MQ: NC, MQ: JE, and NC: JE.

Compositions comprising one or more of prednicarbate, N-palmitoyl-D-threonine and 3,3-difluoro-5-alpha-androstan-17-beta-yl-acetate and a pharmaceutically acceptable carrier are contemplated. The term “pharmaceutically acceptable carrier” refers to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds described herein.

The compositions described herein may be administered orally, gastrointestinally, rectally, or topically on skin or mucosa surfaces. Administration can be in the form of a single bolus dose, or may be, for example, by a continuous perfusion pump. In some embodiments, the composition described herein comprises a disintegrant, a filler, a glidant, or a preservative. In some embodiments, the compositions described herein is formulated as a capsule. Suitable capsules include both hard shell capsules or soft-shelled capsules. Any lipid-based or polymer-based colloid may be used to form the capsule. Exemplary polymers useful for colloid preparations include gelatin, plant polysaccharides or their derivatives such as carrageenans and modified forms of starch and cellulose, e.g., hypromellose. Optionally, other ingredients may be added to the gelling agent solution, for example plasticizers such as glycerin and/or sorbitol to decrease the capsule's hardness, coloring agents, preservatives, disintegrants, lubricants and surface treatment.

In some embodiments, the composition is free from (i) compounds having a molecular weight of greater than 10 kD and (ii) protein/DNA/RNA compounds. In some embodiments, the composition is free from one or more of short-chain fatty acids, polyunsaturated fatty acids, and exopolysaccharides.

### Methods of Treatment

In one aspect, described herein is method of reducing proinflammatory cytokine expression in a subject in need thereof, comprising administering a composition comprising one or more of prednicarbate, N-palmitoyl-D-threonine and 3,3-difluoro-5-alpha-androstan-17-beta-yl-acetate and a pharmaceutically acceptable carrier to the subject in an amount effective to reduce proinflammatory cytokine expression in the subject. Exemplary proinflammatory cytokines include, but are not limited to, IL-1a, IL-1b, IL-6, IL-18, IL-17, IL-8, IL-12, IL-10, IFN-γ (and other interferons), IL-2, IL-11, granulocyte-macrophage colony stimulating factor (GM-CSF), TNF-α, IL-4, TGF-β, IL-22, IL-7, IL-1R, IL-3, IL-5, IL-23, IFN-β1a, and IL-9.

In another aspect, described herein is a method of reducing inflammation in a subject in need thereof, comprising administering a composition comprising one or more of prednicarbate, N-palmitoyl-D-threonine and 3,3-difluoro-5-alpha-androstan-17-beta-yl-acetate and a pharmaceutically acceptable carrier to the subject in an amount effective to reduce inflammation. In some embodiments, the method reduces inflammation in tissues of the subject.

In another aspect, described herein is a method of treating an inflammatory disorder in a subject in need thereof, comprising administering a composition comprising one or more of prednicarbate, N-palmitoyl-D-threonine and 3,3-difluoro-5-alpha-androstan-17-beta-yl-acetate and a pharmaceutically acceptable carrier to the subject in an amount effective to treat the inflammatory disorder. The term “inflammatory disorder” refers to a disease or condition characterized by aberrant inflammation (e.g., an increased level of inflammation compared to a control such as a healthy person not suffering from a disease). Exemplary inflammatory disorders include, but are not limited to, sepsis, dry eye disease, peritonitis, allergy, atopy, asthma, an autoimmune disorder, an autoinflammatory disease, a hypersensitivity, pediatric allergic asthma, allergic asthma, inflammatory bowel disease, Celiac disease, Crohn's disease, colitis, ulcerative colitis, collagenous colitis, lymphocytic colitis, diverticulitis, irritable bowel syndrome, short bowel syndrome, stagnant loop syndrome, chronic persistent diarrhea, intractable diarrhea of infancy, Traveler's diarrhea, immunoproliferative small intestinal disease, chronic prostatitis, postenteritis syndrome, tropical sprue, Whipple's disease, Wolman disease, arthritis, rheumatoid arthritis, Behçet's disease, uveitis, pyoderma gangrenosum, erythema nodosum, traumatic brain injury, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, bullous pemphigoid, sarcoidosis, ichthyosis, Graves ophthalmopathy, Addison's disease, Vitiligo, acne vulgaris, pelvic inflammatory disease, reperfusion injury, sarcoidosis, transplant rejection, interstitial cystitis, atherosclerosis, and atopic dermatitis.

In some embodiments, the inflammatory disorder is an autoimmune disorder. Exemplary autoimmune disorders include, but are not limited to, acute disseminated encephalomyelitis (ADEM), acute necrotizing hemorrhagic leukoencephalitis, Addison's disease, agammaglobulinemia, alopecia areata, amyloidosis, ankylosing spondylitis, anti-GBM/anti-TBM nephritis, antiphospholipid syndrome (APS), autoimmune angioedema, autoimmune aplastic anemia, autoimmune dysautonomia, autoimmune hemolytic anemia, autoimmune hepatitis, autoimmune hyperlipidemia, autoimmune immunodeficiency, autoimmune inner ear disease (AIED), autoimmune myocarditis, autoimmune oophoritis, autoimmune pancreatitis, autoimmune retinopathy, autoimmune thrombocytopeniarpura (ATP), autoimmune thyroid disease, autoimmune urticarial, axonal & neuronal neuropathies, Balo disease, Behcet's disease, bullous pemphigoid, cardiomyopathy, Castleman disease, celiac disease, Chagas disease, chronic inflammatory demyelinating polyneuropathy (CIDP), chronic recurrent multifocal ostomyelitis (CRMO), Churg-Strauss syndrome, cicatricial pemphigoid/benign mucosal pemphigoid, Crohn's disease, Cogan's syndrome, cold agglutinin disease, congenital heart block, Coxsackie myocarditis, CREST disease, essential mixed cryoglobulinemia, demyelinating neuropathies, dermatitis herpetiformis, dermatomyositis, Devic's disease (neuromyelitis optica), discoid lupus, Dressler's syndrome, endometriosis, eosinophilic esophagitis, eosinophilic fasciitis, erythema nodosum, experimental allergic encephalomyelitis, Evans syndrome, fibrosing alveolitis, giant cell arteritis (temporal arteritis), giant cell myocarditis, glomerulonephritis, Goodpasture's syndrome, granulomatosis with polyangiitis (GPA), Graves' disease, Guillain-Barre syndrome, Hashimoto's encephalitis, Hashimoto's thyroiditis, hemolytic anemia, Henoch-Schonlein purpura, herpes gestationis, hypogammaglobulinemia, idiopathic thrombocytopenia purpura (ITP), IgA nephropathy, IgG4-related sclerosing disease, immunoregulatory lipoproteins, inclusion body myositis, interstitial cystitis, juvenile arthritis, juvenile idiopathic arthritis, juvenile myositis, Kawasaki syndrome, Lambert-Eaton syndrome, leukocytoclastic vasculitis, lichen planus, lichen sclerosus, ligneous conjunctivitis, linear IgA disease (LAD), lupus (systemic lupus erythematosus), chronic Lyme disease, Meniere's disease, microscopic polyangiitis, mixed connective tissue disease (MCTD), Mooren's ulcer, Mucha-Habermann disease, multiple sclerosis, myasthenia gravis, myositis, narcolepsy, neuromyelitis optica (Devic's), neutropenia, ocular cicatricial pemphigoid, optic neuritis, palindromic rheumatism, PANDAS (pediatric Autoimmune Neuropsychiatric Disorders Associated with Streptococcus), paraneoplastic cerebellar degeneration, paroxysmal nocturnal hemoglobinuria (PNH), Parry Romberg syndrome, Parsonnage-Turner syndrome, pars planitis (peripheral uveitis), pemphigus, peripheral neuropathy, perivenous encephalomyelitis, pernicious anemia, POEMS syndrome, polyarteritis nodosa, type I, II, & III autoimmune polyglandular syndromes, polymyalgia rheumatic, polymyositis, postmyocardial infarction syndrome, postpericardiotomy syndrome, progesterone dermatitis, primary biliary cirrhosis, primary sclerosing cholangitis, psoriasis, psoriatic arthritis, idiopathic pulmonary fibrosis, pyoderma gangrenosum, pure red cell aplasia, Raynaud's phenomenon, reactive arthritis, reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, Sjogren's syndrome, sperm & testicular autoimmunity, stiff person syndrome, subacute bacterial endocarditis (SBE), Susac's syndrome, sympathetic ophthalmia, Takayasu's arteritis, temporal arteritis/giant cell arteritis, thrombocytopenia purpura (TTP), Tolosa-Hunt syndrome, transverse myelitis, type 1 diabetes, asthma, ulcerative colitis, undifferentiated connective tissue disease (UCTD), uveitis, vasculitis, vesiculobullous dermatosis, vitiligo, and Wegener's granulomatosis.

According to the methods provided herein, the subject is administered an effective amount of one or more of the compounds herein. The term effective amount is defined as any amount necessary to produce a desired physiologic response (e.g., reduction of inflammation, infection, or dysbiosis). Effective amounts and schedules for administering the agent may be determined empirically by one skilled in the art. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, anaphylactic reactions, and the like. Generally, the dosage will vary with the age, condition, sex, type of disease, the extent of the disease or disorder, route of administration, or whether other drugs are included in the regimen, and can be determined by one of skill in the art. The dosage can be adjusted by the individual physician in the event of any contraindications. Dosages can vary and can be administered in one or more dose administrations daily, for one or several days. Guidance can be found in the literature for appropriate dosages for given classes of pharmaceutical products. For example, for the given parameter, an effective amount will show an increase or decrease of at least 5%, 10%, 15%, 20%, 25%, 40%, 50%, 60%, 75%, 80%, 90%, or at least 100%. Efficacy can also be expressed as “-fold” increase or decrease. For example, a therapeutically effective amount can have at least a 1.2-fold, 1.5-fold, 2-fold, 5-fold, or more effect over a control. The exact dose and formulation will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 20th Edition, Gennaro, Editor (2003), and Pickar, Dosage Calculations (1999)).

For prophylactic use, a therapeutically effective amount of the composition described herein are administered to a subject prior to or during early onset (e.g., upon initial signs and symptoms of an inflammatory disease or an autoimmune disease). Therapeutic treatment involves administering to a subject a therapeutically effective amount of the agents described herein after diagnosis or development of disease. Thus, in another aspect, a method of treating a disease (e.g., an inflammatory disease) in a subject in need thereof is provided.

The terms “subject,” “patient,” “individual,” etc. are not intended to be limiting and can be generally interchanged. That is, an individual described as a “patient” does not necessarily have a given disease, but may be merely seeking medical advice.

As used herein, “treating” or “treatment of” a condition, disease or disorder or symptoms associated with a condition, disease or disorder refers to an approach for obtaining beneficial or desired results, including clinical results. Beneficial or desired clinical results can include, but are not limited to, alleviation or amelioration of one or more symptoms or conditions, diminishment of extent of condition, disorder or disease, stabilization of the state of condition, disorder or disease, prevention of development of condition, disorder or disease, prevention of spread of condition, disorder or disease, delay or slowing of condition, disorder or disease progression, delay or slowing of condition, disorder or disease onset, amelioration or palliation of the condition, disorder or disease state, and remission, whether partial or total. “Treating” can also mean prolonging survival of a subject beyond that expected in the absence of treatment. “Treating” can also mean inhibiting the progression of the condition, disorder or disease, slowing the progression of the condition, disorder or disease temporarily, although in some instances, it involves halting the progression of the condition, disorder or disease permanently. As used herein the terms treatment, treat, or treating refers to a method of reducing the effects of one or more symptoms of a disease or condition characterized by expression of the protease or symptom of the disease or condition characterized by expression of the protease. Treatment can refer to a 10%, 20%, 30%, 40%, 50%, 60%, 70%, 80%, 90%, or 100% reduction in the severity of an established disease, condition, or symptom of the disease or condition (e.g., inflammation). For example, a method for treating a disease is considered to be a treatment if there is a 10% reduction in one or more symptoms of the disease in a subject as compared to a control. Thus, the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. Further, as used herein, references to decreasing, reducing, or inhibiting include a change of 10%, 20%, 30%, 40%, 50%, 60%, 70%, 80%, 90% or greater as compared to a control level and such terms can include but do not necessarily include complete elimination of the symptoms or disease.

### EXAMPLES

### Example 1—Metabolites that Suppress Both TLR Agonists and NLRP3 Inflammasome Activation

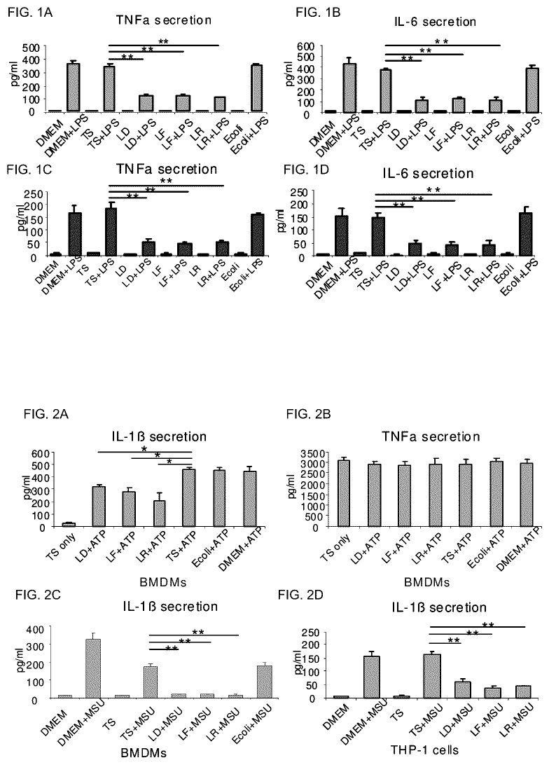

To explore the metabolites that suppress both TLR agonists and NLRP3 inflammasome activation, we cultured commensal bacteria, Lactobacillus rhamnosus (LR), lactobacillus fermentum (LF), and lactobacillus dehbruckii (LD), and collected supernatants and tested if any regulatory metabolites existed. To facilitate subsequent identification of a metabolite, the supernatants were separated into two fractions via passage through a 10 kDa filter (termed as >10 kDa and <10 kDa fractions). We then used the fractions to pre-treat mouse bone marrow derived macrophages (BMDMs), and human macrophages (THP-1), followed by treatment with LPS, a TLR4 agonist. We found the fraction (<10 kD) from all three bacteria strains inhibited macrophage production of pro-inflammatory cytokines (IL-6 and TNFα) (FIG. 1).

We further tested if they could inhibit NLRP3 inflammasome activation, an important inflammation initiator for innate immune cells. To do so, we primed BMDMs and THP-1s with LPS, then pre-treated them with the <10 kDa fraction. Lastly, we stimulated the cells with an NLRP3 activator, ATP or Monosodium urate Crystals (MSU). When cells are primed with LPS, they will express intracellular pro-IL-1β and secrete TNFα, but not mature IL-1β. Only after they are stimulated with inflammasome activators, can they secrete IL-1β. We found, that IL-1β secretion in response to NLRP3 inflammasome activators (ATP or MSU) was significantly reduced after treatment with the fractionated supernatants from the three strains. (FIG. 2).

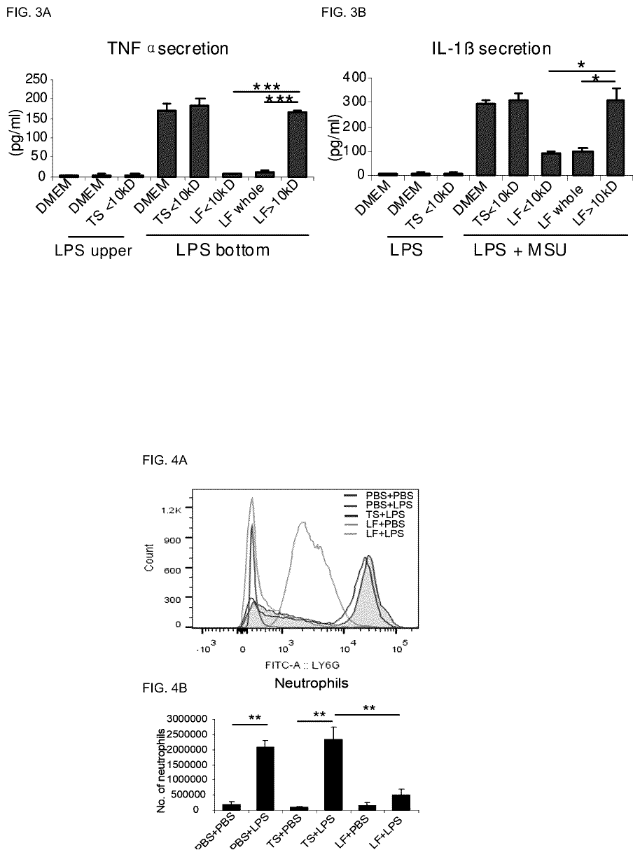

Commensal bacteria are non-invading bacteria that habit the host gastrointestinal tract without causing harm. Therefore, the metabolite(s) must be able to cross the epithelium to mediate their functions. Therefore, we used human epithelial cells (Caco-2) to mimic the tight junctions formed by intestinal epithelial cells. We used a transwell system that has an upper chamber and lower chamber. The upper chamber was cultured with Caco-2 cells for 2 weeks, to allow tight junctions to form, while the lower chamber contained THP-1 macrophages. We then measured the ability of the <10 kDa fraction to cross intestinal epithelium with the tight junctions without disrupting the integrity. We found that the bacterial factors/metabolites can pass through the epithelium to suppress production of TNFα and IL-1β production by the macrophages (FIG. 3). To determine if the <10 kD fraction suppresses LPS-induced inflammatory responses in vivo, we intraperitoneally injected the <10 kDa fraction into C57/Bl6 mice followed by LPS and then analyzed peritoneal numbers of neutrophils by flow cytometry. We found the number of neutrophils were significantly reduced in mice that were pre-treated with the <10 kD fraction (FIG. 4). These data support LF, LD, LR suppress inflammatory response in vivo by metabolite(s) they secrete.

To determine if the responsible factors were protein, DNA/RNA or lipids, we treated the <10 kD fraction with RNaseA to digest RNA, DNase I to digest DNA, boiling to denature proteins, or with chloroform extraction to remove lipids/fatty acids and then incubated with B6 Macrophages followed by LPS stimulation. We found that the inhibitory activity in the <10 kDa fractions was unaffected by treatments with RNaseA, DNase I treatment or boiling, indicating that they are not RNA, DNA and protein in nature. In contrast, the chloroform-extracted fractions failed to inhibit the activation of macrophages by LPS (data not shown). These data suggest that the inhibitory factors present in the <10 kDa fractions may be lipids. The control E. coli culture supernatant did not have any suppressive activity.

Next, the active metabolite(s) in the <10 kD fraction was identified. To do so, we performed metabolomics analysis by mass spectrometry on <10 kD fraction from LF, LD, and LR having suppressive inflammatory capabilities. We then compared the lists of metabolites from LF, LD and LR to find commonalities between all three. We compared the characterized metabolites to all known indole, niacin, and SCFAs derivatives. The database search indicated none of the known derivates to be present in our characterized list of metabolites from the <10 kD fraction of LF, LD, and LR. Furthermore, we proceeded to find commonalities between the three characterized <10 kD fraction. We narrowed down our search by picking metabolites that were present at high concentrations and no known toxic effects. We were able to identify three metabolites: prednicarbate (“MQ”), 3,3-Difluoro-5-alpha-androstan-17-beta-yl acetate (“JE”) and N-Palmitoyl-D-Threonine (2˜{R},3˜{S})-2-(hexadecanoylamino)-3-hydroxybutanoic acid) (“NC”).

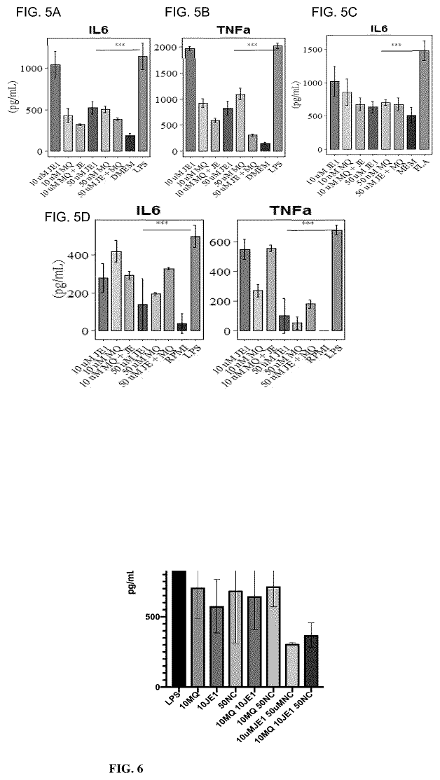

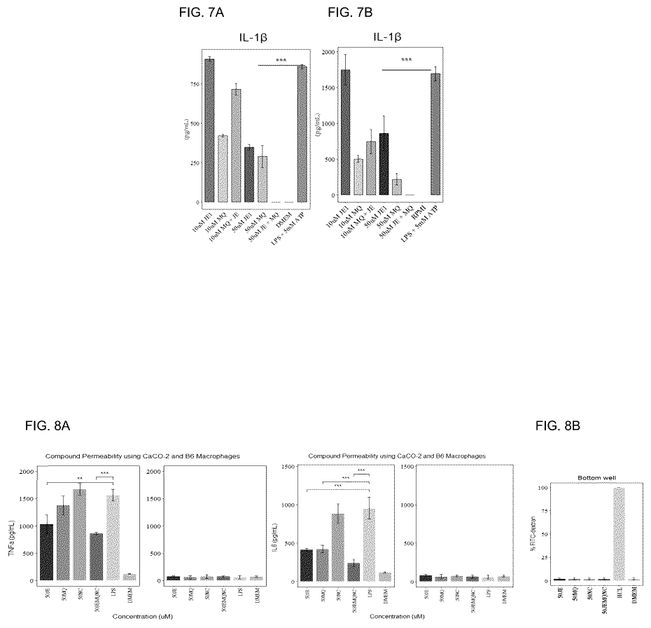

Next, the metabolites were tested individually to assess their regulatory effects on mouse macrophages and human THP-1 macrophages. Similar to the bacterial <10 kD supernatant fraction, both JE and MQ inhibited LPS-stimulated macrophage production of TNFa and IL-6 (FIG. 5), indicating it suppresses TLR4 agonist-induced production of pro-inflammatory cytokines by macrophages. We further found that combination of them led to higher inhibition (FIG. 6). Additionally, JE and MQ also inhibited IL-β production stimulated by LPS and ATP, demonstrating that they inhibit NLRP3 inflammasome activation (FIG. 7)

As stated before, commensal bacteria are non-invading and the metabolites must be able to cross the epithelium to mediate their functions and our earlier data confirmed <10 kD fraction pertained this ability. Therefore, we used the trans-well system to test if the identified metabolites were able to cross the epithelium. As we did previously, Caco-2 cells were culture for 2 weeks, to allow tight junctions to form. While the lower chamber contained C57BL/6 mouse BMDMs. We then measured the ability for NC, MQ and JE to cross the tight junctions without disrupting the integrity of the tight junctions. We did each separately and combined as well. We found that NC, JE and MQ can pass through tight junctions of epithelial cells to suppress TNFα production by THP1 cells (FIG. 8).

Having shown that compound JE was capable of suppressing macrophage activation upon LPS and ATP/MSU stimulation in vitro, we further validated the suppressive role of JE in vivo upon LPS stimulation. Specifically, we wanted to determine whether it could suppress neutrophil recruitment to the peritoneal cavity after intraperitoneal injections of LPS stimuli, which serves as a sepsis model.

In humans and other mammals, the peritoneal cavity is a large, hollow space within the body. The peritoneum plays as a key role in local defense against pathogenic invasion. These peritoneal defense mechanisms rely on the activation local immune cells as well the recruitment of circulating immune cells. Specifically, resident macrophages control the early stage of neutrophil recruitment during tissue inflammation or pathogenic invasion.

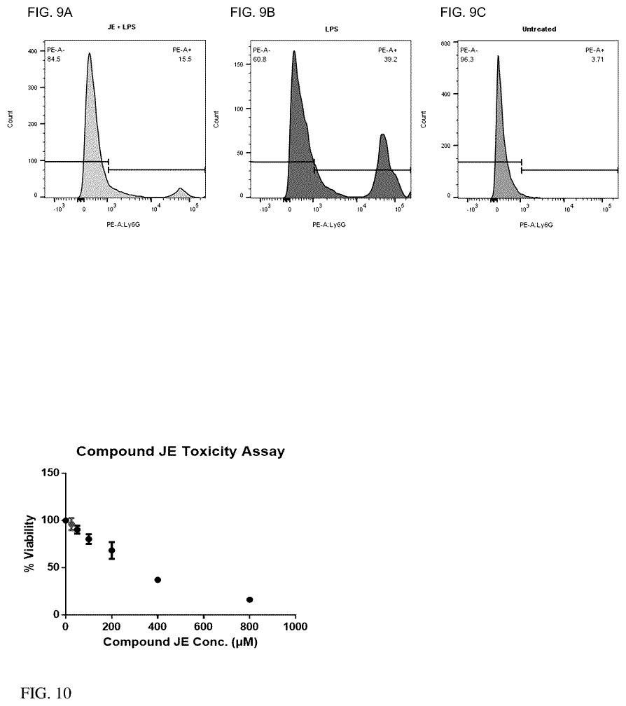

In order to determine if JE had any effect on this neutrophil recruitment, we first intra-peritoneally injected the metabolite into mice for two consecutive days. On day 3 we intraperitoneally injected LPS to stimulate neutrophil recruitment. After 4 hours, mice were sacrificed and the peritoneal cavities were washed with DMEM. The recruited neutrophils present in the peritoneal lavage fluid were quantified by flow cytometry using neutrophil marker Ly6G. Flow cytometry analysis showed that peritoneal neutrophil recruitment was reduced in mice that were pretreated with compound JE as compared to those that received PBS (FIG. 9)

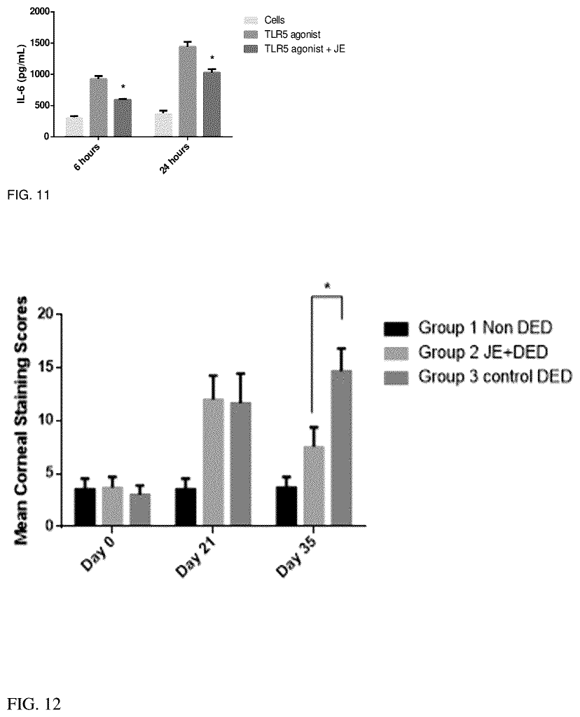

To test if JE has any toxicity, we first incubated murine macrophages with JE at different concentrations at 37° C. for two days, and determined viability of the cells. Although higher concentrations >100 μM reduced the viability, JE at 50 μM or below did not show significant reduction (FIG. 11). Further we injected the JE at 50 μM twice daily via I.P. for 3 or 5 days and then determined ALT levels in sera and examined the morphology in key organs, liver, kidney, spleen, intestines etc. We found no significant higher levels of ALT in the group treated with JE than the control group at both time points. We did not see any tissue damage in the organs by histology (data not shown).

To test if JE could be used as a therapeutic agent, we tested its effects on a dry eye disease model. Dry eye disease is characterized by inflammation in ocular surface. First, we tested if JE can inhibit production of pro-inflammatory cytokine IL-6 by corneal epithelial cells stimulated by TLR5 agonist flagellin. Thus, SV 40 immortalized human corneal epithelial cells (HCECs) were with JE at 50 μM overnight and they are subsequently exposed to TLR5 agonists (flagellin isolated from Pseudomonas aeruginosa) for 6 or 24 hours. The cell culture supernatants were collected and used for ELISA to determine the concentrations of IL-6. We found that HCECs produced large amounts of inflammation cytokine IL-6 in response to TLR5 agonist flagellin from Pseudomonas aeruginosa. Probiotic bacteria-derived metabolite (JE) suppressed the TLR5 agonist-induced IL-6 production (FIG. 11).

We further tested if JE reduced corneal tissue damage in a murine DED model. Thus, normal C57BL/6J mice were maintained in relative humidity 60% to 80%, no airflow at 21-23° C. as normal control (Group 1). The experimental dry eye was induced by using an Intelligently Controlled Environmental System (ICES). Dry eye mice (Group 2 and Group 3) were housed in ICES with relative humidity at 13+4%, airflow of 2.2+0.2 m/s, and temperature of 21-23° for 3 weeks. Group 2 mice were treated with 10 μL JE at 50 μM 0.35% (v/v) DMSO in PBS and group 3 (vehicle control) were treated with 10 μL of 0.35% (v/v) DMSO in PBS, 4 time/day topically for 14 days. Corneal fluorescein staining was evaluated at week 0, 3 and 5. The stained area was assessed according to a standardized grading system ranging from 0 to 4, with the corneal surface divided into five regions. Then the total scores from the five regions were analyzed. After mice developed dry eye conditions (3 weeks), JE treatment significantly reduced corneal staining compared with PBS control on day 35 (FIG. 12). The data strongly support that JE (3,3-Difluoro-5-alpha-androstan-17-beta-yl acetate) can be used as therapeutic agent for non-infectious inflammatory diseases.

### Methods:

Commensal bacterial culture and supernatant collection. These four Lactobacillus strains include delbrueckii (LD), Lactobacillus rhamnosus (LR), Lactobacillus fermentum (LF) and Streptococcus oralis (SO). Bacteria were cultured separately in tryptic soy (TS) broth, pH: 6.0-6.4 at 37° C., 5% CO₂ for three weeks until they reached an OD600 of 1.0. The bacteria cultures were then centrifuged at 10,000 rpm for 15 minutes, collected, and applied through a 0.22 μm filter (Millex) to remove any whole cells. Bacterial supernatants were stored at −20° C.

Cell Culture: Multiple macrophage cell lines were used in these experiments. Primary murine BMDMs were differentiated through isolation and treatment with L929 conditioned medium. C57BL/6 macrophages and BMDMs were seeded in complete DMEM (10% FBS+1% Penicillin-Streptomycin) in 24-well plates at concentrations of 5×10⁵ and 2.5×10⁵, respectively. THP-1 cells were maintained and grown in complete RPMI-1640 medium. Cells were differentiated by stimulating with PMA (100 nM) for three hours at 37° C. with 5% CO-2 until adherent. They were then collected and plated at a density of 2.5×105 cells per well in 24-well plates with serum-free RPMI and incubated overnight.

Treatment and denaturation of bacterial supernatant: Bacterial supernatant (LD, SO, LR or LF) was treated with either RNase A (5 U/mL, Thermo Scientific), DNase I (5 U/mL, Thermo Scientific), or boiled at 100° C. for 10 minutes to denature proteins.

Bacterial fractionation by ultracentrifugation: Bacteria supernatants (LD, SO, LR or LF) and TS bacterial culture media control were separated into >10 KDa and <10 KDa fractionates using Amicon Ultra 38 centrifugal filters (Millipore) and spinning at 5000 rpm for 30 minutes.

### Example 2—Metabolite JE Suppressed Production of TNF-α and IL-6 as Well as Caspase-1 Activation

Murine macrophages, both RAW 264.7 macrophages and bone marrow-derived macrophages (BMDMs), were seeded and then pretreated overnight with the metabolite JE. The following day, the macrophages were stimulated with one of TLR agonists, including LPS, FLA-ST, ODN, Pam3SCK4, Poly: IC LMW, FSL, and Imiquimod for 3 hours, then the production of TNF-α, and IL-6 in the culture supernatants was measured by ELISA.

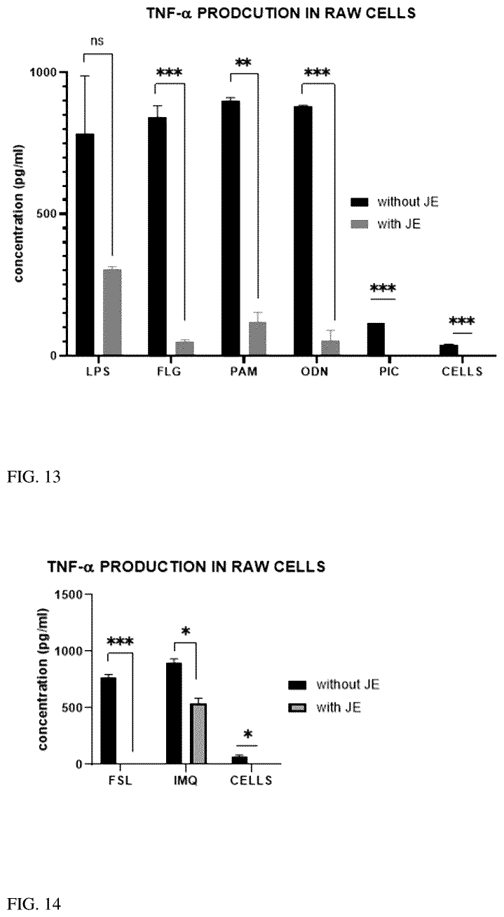

It was observed that when murine RAW 264.7 macrophages were treated with the metabolite ‘JE’ before being stimulated with TLR agonists, the production of TNF-α and IL-6 was significantly reduced compared to macrophages that were only exposed to media with TLR agonists. Pretreatment with the metabolite “JE” completely abolished TNF-α secretion when macrophages were stimulated with TLR2/6 agonist, FSL-1 or TLR-3 agonist Poly: IC LMW. Even though stimulation of macrophages with Poly: IC did not result in robust TNF-α production in cells not treated with JE, inhibition by JE is apparent (FIG. 13 and FIG. 14).

Macrophages pretreated with JE exhibited a 4-fold decrease in TNF-α production upon stimulation with TLR 1/2 agonist, PAM-3CSK and TLR 5 agonist, FLG-ST compared to untreated macrophages. On the other hand. in case of stimulation with TLR4 agonist, LPS and TLR7 agonist, Imiquimod, there is still TNF-α production even after inhibition by JE. However, JE still reduced TNF-α production (FIG. 13 and FIG. 14).

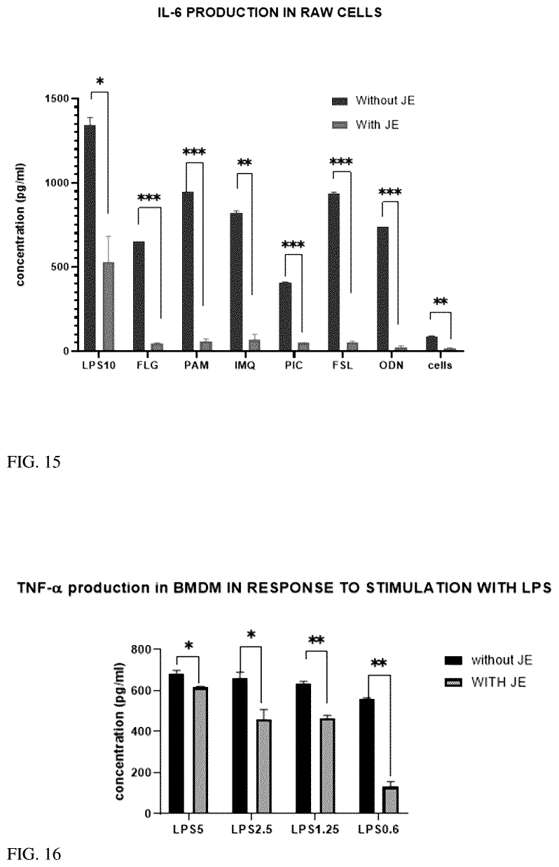

Murine RAW 264.7 cells exhibit a 4-fold decrease in IL-6 secreted by macrophages stimulated with LPS after pretreatment with JE relative to untreated macrophages. Even though IL-6 levels in pretreated cells stimulated with LPS remain slightly elevated, IL-6 inhibition by JE is significant, as shown in FIG. 15.

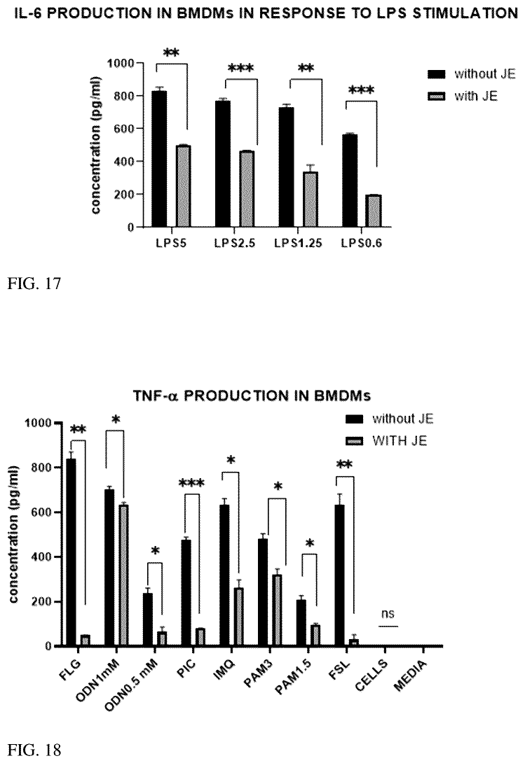

Unlike RAW 264.7, when I stimulated BMDMs with LPS at a concentration of 10 ng/ml, JE failed to decrease TNF-α or IL-6 production (data not shown). Therefore, I stimulated them with two-fold dilution of the concentration I used to stimulate RAW 264.7. Significant inhibition of TNF-α secretion by BMDMS occurred at 1.25 ng/ml and 0.6 ng/ml of LPS (FIG. 16). On the other hand, JE could reduce IL-6 secretion by BMDMs at 5 ng/ml of LPS as depicted in FIG. 17.

Similar to treatment of RAW 264.7 macrophages with TLR agonists, I pretreated BMDMs with JE then stimulated them with FLG, ODN, PIC, Imiquimod, PAM-3SCK, and FSL-1. I stimulated these macrophages with two-fold dilutions of ODN and PAM-3SCK because JE did not significantly reduce TNF-α production in a previous experiment (data not shown). After 3 hours, Supernatants were collected, TNF-α and IL-6 were quantified by ELISA. Overall, JE decreased both IL-6 and TNF-α secretion by BMDMs upon stimulation with TLR agonists as shown in FIG. 17 and FIG. 18, However, the data show that JE has a greater effect on reducing IL-6 production in BMDMs stimulated with ODN compared to those stimulated with TNF-α.

Next, we explored how JE inhibits caspase-1 activation as well as IL-1β production. As stated above, NLRP3 activation adopts a two-step dogma: priming with LPS, followed by activation by ATP, nigericin, or MSU crystals. This results in NLRP3 inflammasome activation and generation of activated caspase-1 through proximity-induced catalytic cleavage of caspase-1 and conversion of pro-IL-1β into IL-1β. To achieve that, JE was used to pretreat BMDMs overnight at 37° C., 5% CO2; the next day, macrophages were primed with LPS for 4 hours, then stimulated with ATP for an hour. I collected the supernatant and measured IL-1β by ELISA.

In an attempt to determine whether JE inhibits the priming or the activation step, BMDMs were stimulated with LPS, then the metabolite JE was used to treat the cells overnight at 37° C., 5% CO₂. The following day, macrophages were stimulated with ATP for an hour, then, supernatant was collected to measure IL-1β by ELISA. PBS was used as a control to JE in this experiment. In both conditions, I did a western blot on caspase-1 to elucidate whether JE inhibits caspase-1 activation or not.

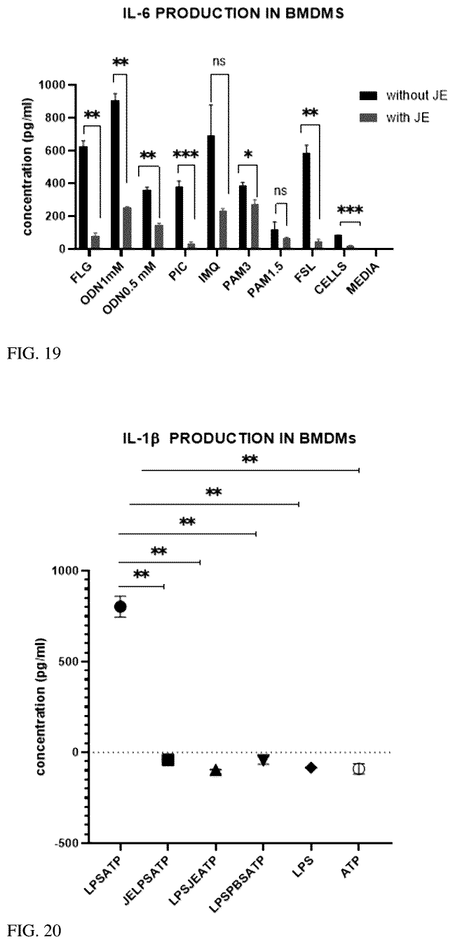

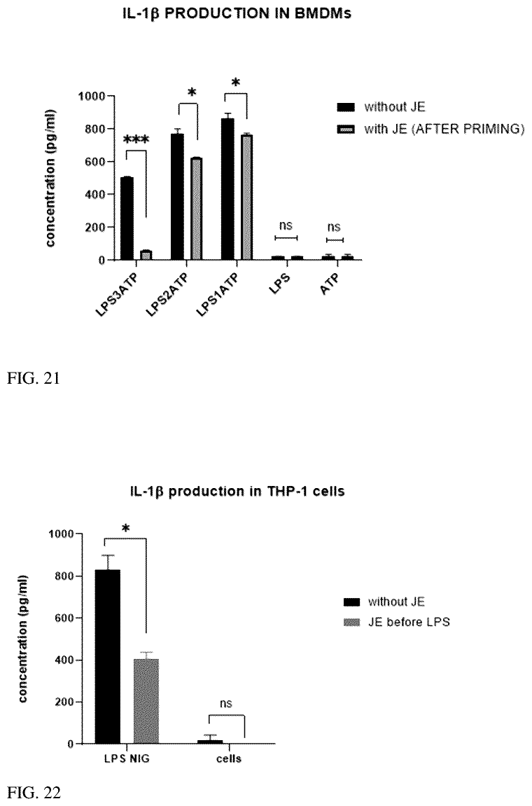

The metabolite JE inhibited the priming step and completely abolished IL-β upon activation of NLRP3 inflammasome (FIG. 20). It was also evident by the absence of active caspase-1 in the supernatant on western blot analysis (data not shown). To determine whether JE inhibits the activation signal, instead of pretreating the macrophages with JE overnight. BMDMs were treated with the metabolite JE for 1 hour, 2 hours, and 3 hours after priming with LPS for 4 hours; then 5 mM of ATP was. The data show that treating macrophages with JE for 3 hours after priming with LPS can significantly decrease IL-1β production upon NLRP3 activation with ATP. On the other hand, treating the cells 1 hour or 2 hours with JE did not have the same effect (FIG. 21).

We similarly tested THP-1 cells as described above. THP-1 cells were differentiated with PMA to their macrophage phenotype, then I pretreated THP-1 cells with the metabolite JE overnight at 37° C., 5% CO₂; the next day, I primed the macrophages with LPS for 4 hours, then I activated them with ATP or nigericin for an hour. I collected the supernatant and measured IL-1β by ELISA.

To determine whether JE inhibits the priming or the activation step, THP-1 cells were stimulated with LPS, then, the metabolite JE was added to pretreat the cells overnight at 37° C., 5% CO₂. The following day, macrophages were activated with ATP or nigericin for an hour, then the supernatant was collected to measure IL-1β by ELISA.

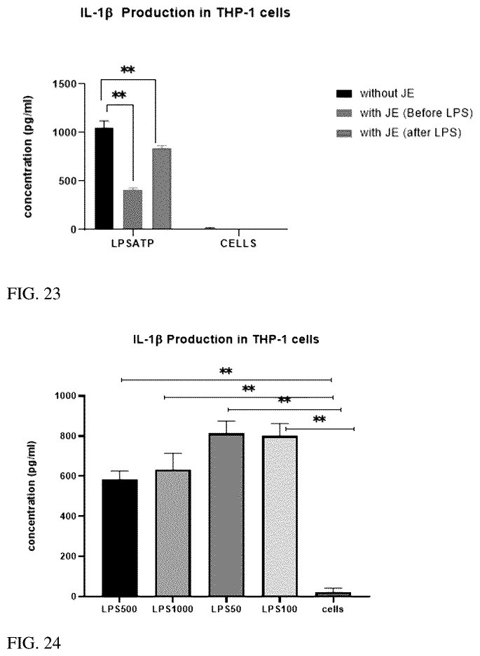

Our data demonstrate that the metabolite ‘JE’ could decrease IL-1β production by THP-1 macrophages upon NLRP3 activation when pretreated with JE, i.e., when JE is added before priming with LPS. Therefore, JE is capable of inhibiting the priming step of inflammasome, as demonstrated in FIG. 22, however, when I treated the macrophages with JE, i.e., after priming with LPS and before Adding ATP, we still detected robust production IL-1β (FIG. 23). This is due to the capability of THP-1 cells to induce NLRP3 inflammasome assembly and caspase-1 activation, resulting in IL-1β production by engaging in an alternative pathway (FIG. 24)

### Example 3—Metabolite JE in a Dry Eye Disease (DED) Model

The DED mouse model comprises three groups; each with three wild-type C57BL/6J mice groups. Group 1 served as a standard control, and the mice were maintained in relative humidity of 60% to 80%, with no airflow at 21-23° C. The experimental dry eye was induced by using an Intelligently Controlled Environmental System (ICES). Dry eye mice (Group 2 and Group 3) were housed in ICES with relative humidity at 13+4%, airflow of 2.2+0.2 m/s, and temperature of 21-23° for three weeks. Group 2 mice were treated with 10 μl JE at 50 μM 0.35% (v/v) DMSO in PBS, and group 3 (vehicle control) were treated with 10 μl of 0.35% (v/v) DMSO in PBS, 4 times/day topically for 14 days. Room humidity and chamber humidity were tracked daily during the period of inducing the disease.

The data show that mice fully developed dry eye disease in around three weeks, as indicated by fluorescein punctate staining (data not shown). This pattern of staining points to significant corneal damage and inflammation. Importantly, administering eye drops containing the metabolite ‘JE’ four times a day for 14 days led to significant improvements in the disease. This was demonstrated by reduced corneal fluorescein staining scores and a decrease in the surface area of corneal damage relative to vehicle control. Next, the IL-6 cytokine expression was measured in corneal samples using RT-qPCR. Isolation of corneal samples was done, and then they were stabilized in RNA later buffer and stored at-80 for RNA purification.

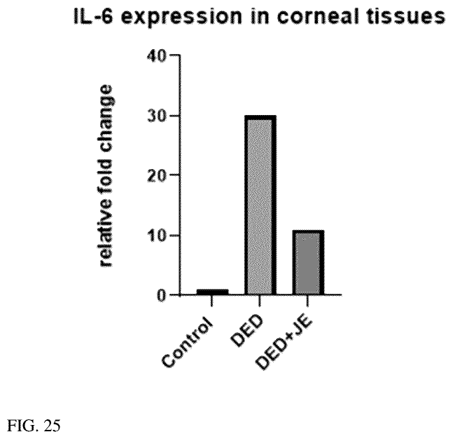

For RNA purifications, samples were homogenized, and RNA extraction was performed. A total of 0.5 μg to 1 μg RNA was yielded. In order to measure the expression of IL-6, RT-qPCR was performed. Strong IL-6 expression was observed in the disease group compared to the control group. When JE eye drops were administered, IL-6 expression was decreased. However, IL-6 expression levels did not return to the baseline (FIG. 25).

### Example 4—MQ Reduced Cytokine Production In Vitro in Response to TLR Agonists

Materials and Methods: Human corneal epithelial cells were grown in culture. Cells were then plated in DMEM. The cells cultures were then kept at 37° C. with 5% CO₂. Once ready, HCECs were then incubated with MQ at a concentration of 50 uM overnight. The following day they were treated with FLA at 200 ng/mL. Samples were treated for 3 hours and then samples were collected. Nuclear and cytoplasmic fractions were obtained using 0.1% NP-40 buffer. Cells were pelleted and the supernatant was discarded. Cells were then treated with 25 μl of NP-40 lysis buffer. Upon addition of the buffer cells were centrifuged for 10 minutes at 3000 rpm at 4° C. The supernatant was collected which is the cytoplasmic fraction and the remaining pellet was the nuclear fraction. The pellet was then washed three times to remove any remaining cytoplasmic fraction.

Both fractions were then treated with 2x SDS loading buffer and the protein was denatured at 100° C. and placed on ice. Proteins were run on a gel at 15 A until the dye front neared the bottom of the gel. Protein was then dry transferred to a membrane. The membrane was blocked in 5% blocking buffer overnight. The following day the membrane was washed three times with PBS-T and anti-mouse p65-NF-kB rabbit polyclonal antibody was added. The membranes were left at room temperature to shake for two hours. Following this the membrane was washed and secondary goat anti-rabbit HRP conjugated antibodies were added and left on the membrane for one hour. Following this the membranes were exposed to 10 mL of chemiluminescent substrate for 10 minutes. The proteins were then visualized using a FluorChem E System (ProteinSimple).

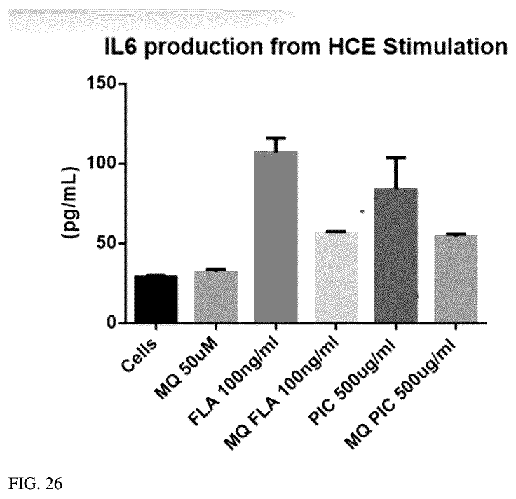

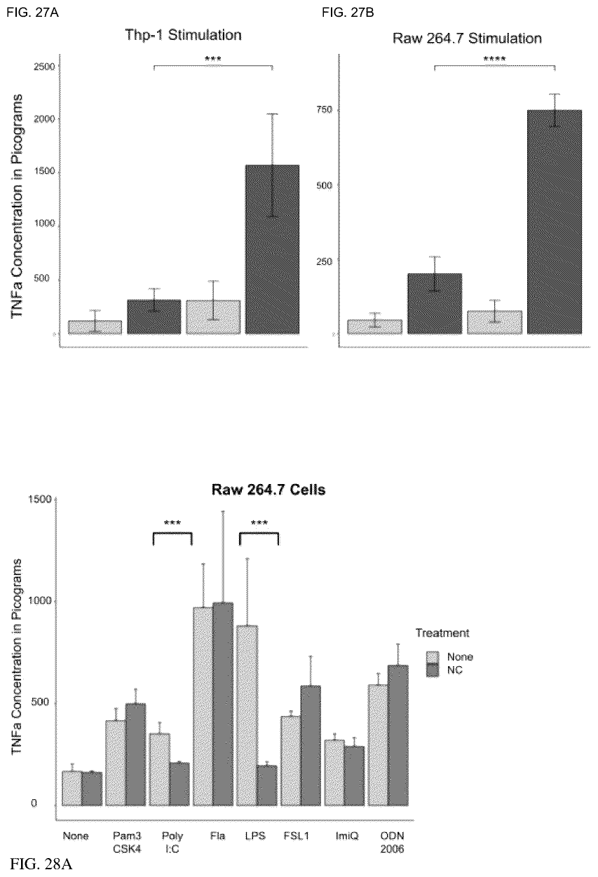

To investigate the effects of MQ on cytokine activity, human corneal epithelial cells (HCECs) were stimulated with various TLR agonists and the production of cytokines was measured via ELISA. HCECs were seeded at a density of 2×10⁵ cells/well and were treated overnight with MQ at a concentration of 50 μM and the following day they were stimulated with Flagellin (FLA) at 100 ng/ml or Poly (I:C) at a concentration of 500 μg/ml. After 4 hours, supernatant was collected and the concentration of IL-6 was determined via ELISA. MQ alone was not found to increase production of IL-6, whereas pre-treatment with MQ reduced the production and secretion of IL6 in HCECs (FIG. 26).

The effects of MQ on various other TLR pathways including: TLR 1/2 (PAM3CSK4), TLR 2/6 (FSL) TLR 4 (Lipopolysaccharide), TLR 7 (Imiquimod), and TLR 9 (ODN2006) was also investigated. Following the same pretreatment and stimulation protocol, and while testing various concentrations, MQ did not exhibit any significant inhibitory effects on cytokine production.

The inhibitory effects on the translocation of NF-kB from the cytoplasm to the nucleus following stimulation with FLA was also assessed. HCECs were treated overnight with MQ at a concentration of 50 μM and the following day they were stimulated with Flagellin (FLA) at 100 ng/ml. After 3 hours, the cells were collected and treated with 0.1% NP40 buffer to separate the nuclear and cytosolic fractions. Western blot analysis was then performed to detect the p65 subunit of the NF-kB protein. The blot showed that MQ inhibited translocation of NF-kB to the nucleus of HCECs during FLA stimulation (data not shown).

### Example 5—NC Reduced Cytokine Release in Response to TLR Agonists In Vitro

To investigate the effects of compound NC, macrophages were stimulated with LPS, an endotoxin associated with gram-negative bacteria. Human and murine macrophage-like cells (THP-1 cells and Raw 264.7 cells, respectively) were plated in 24 well plates at a uniform density of 0.2×106 cells/mL. Following 18 hours of pretreatment with 50 μM metabolite NC, they were stimulated with 10 μg/mL of LPS. After 4-6 hours, supernatant was collected and TNFα was quantified using sandwich ELISA. NC reduces TNFα release from both THP-1 and Raw 264.7 macrophage-like cells in response to TLR4 agonist LPS in vitro. NC did not increase TNFα release in the absence of LPS stimulation. See FIG. 27.

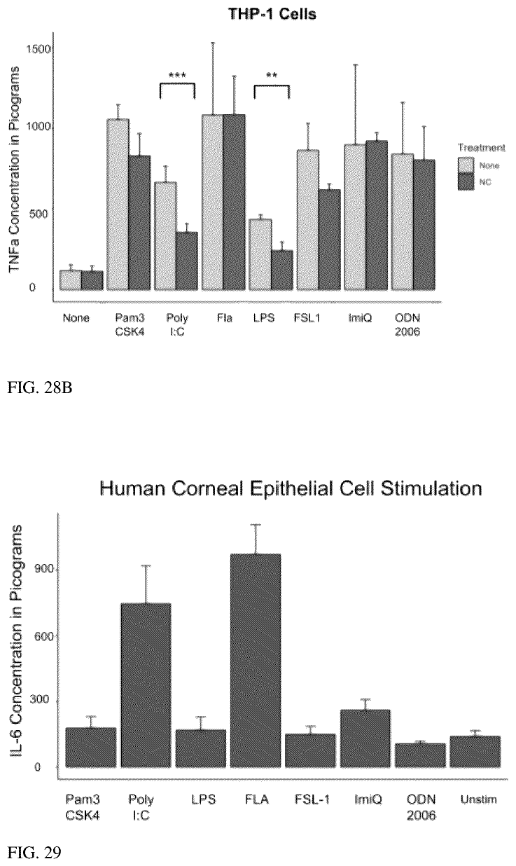

Metabolite NC reduces inflammation in response to TLR4 agonist LPS. To further investigate the specificity of NC's action in TLR signaling, Raw 264.7 (FIG. 28A) and THP-1 (FIG. 28B) cells were once again plated at a uniform density of 0.2×10⁶ cells/mL, treated with 50 μM metabolite NC for 18 hours, and stimulated with agonists for TLR1/2, TLR3, TLR4, TLR5, TLR6/2, TLR7 and TLR9. After 4-6 hours, supernatant was collected and TNFα was quantified via sandwich ELISA.

NC reduced TNFα release in response to TLR3 and TLR4 agonists (Poly I:C and LPS, respectively) in both human and murine macrophages, but did not reduce TNFα release in response to any other TLR agonists (FIGS. 28A and 28B). Interestingly, TLR3 and TLR4 have different locations in the cell. TLR4 is located on the membrane, while TLR3 is located in the endosome. Of all the TLRs, only TLR3 and TLR4 signal through TRIF. Additionally, TRIF is the only adaptor associated with TLR3. Therefore, NC may act through TRIF to reduce TNFα release in response to TLR3 and TLR4 agonists.

Human corneal epithelial cells (HCECs) are a cell type used for models of ocular inflammation. To determine the specificity of HCEC TLR signaling, HCECs were stimulated with various TLR agonists. In this experimental model, HCECs only release IL-6 in response to TLR3 and TLR5 agonists (Poly I:C and Fla, respectively) in vitro (FIG. 29). Therefore, only Poly I:C and Fla were used for HCEC stimulation in subsequent experiments.

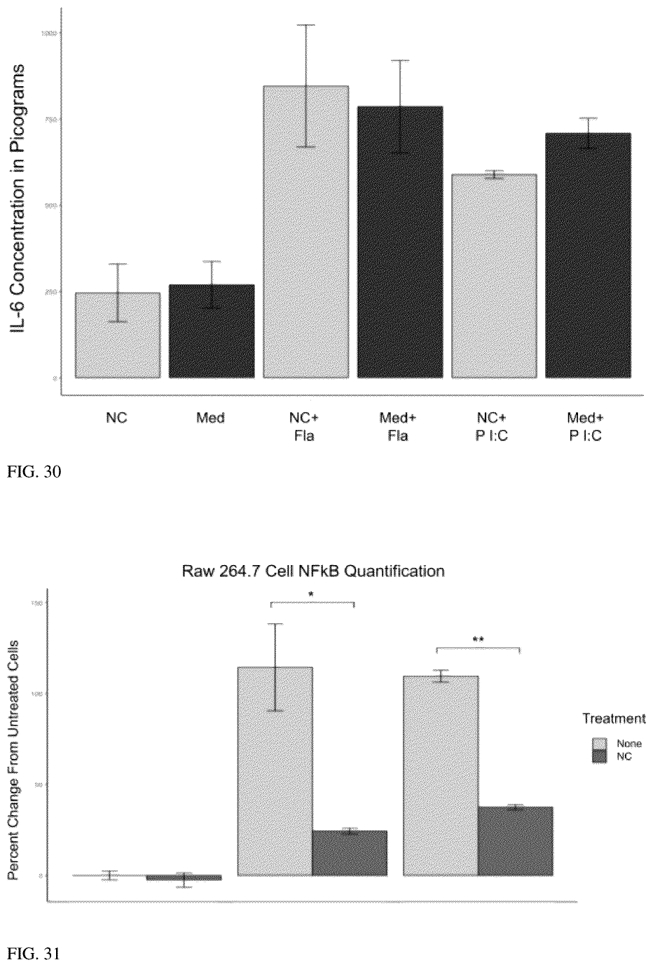

To determine if NC can mitigate TLR-related inflammation, HCECs were plated at a uniform density, incubated with 50 μM metabolite NC for 18 hours, and stimulated with flagellin or Poly I:C. After 6-8 hours, supernatant was collected and IL-6 was quantified. NC does not significantly reduce the release of IL-6 in response to Poly I:C or Fla in HCECs. NC modestly reduced IL-6 release in response to Poly I:C, but this effect was marginal compared to trials in macrophages. FIG. 30.

### Example 6—NC Reduced Inflammation Through NFκB

NC was found to reduce TNFα release in response to TLR3 and TLR4 agonists, indicating that it may act in a TRIF-dependent manner. TRIF can participate in TNFα signaling through two distinct mechanisms. Firstly, TRIF can signal NFκB through TRAF6 to increase TNFα transcription. Secondly, TRIF activation can enhance the translation of TNFα mRNA through prolonged MK-2 activation. To determine whether NC reduces TNFα release by blocking TNFα transcription or translation, we collected cell lysates and quantified the levels of phosphorylated NFκB (ser536). Lysates of Raw 264.7 cells treated with NC prior to stimulation with LPS or Poly I:C contained more phosphorylated NFκB (ser536) than untreated cells (FIG. 31). There was no difference in phosphorylated NFκB (ser536) in unstimulated cells treated with NC.

### Example 7—NC Reduced Cytokine Production In Vitro in Response to TLR Agonists

### Materials and Methods

Bone marrow derived macrophages were collected from adult mice (6-8 weeks old). Femurs were obtained and cut with scissors. The connecting muscles were removed and the bones were flushed with PBS. PBS was collected and cells were counted and adjusted to the desired concentrations. Cells were then plated in DMEM supplemented with Macrophage Colony Stimulating Factor (M-CSF) at 10 ng/ml. The cells cultures were then kept at 37° C. with 5% CO₂ for 7 days, with the media being replaced every two days. After 7 days the cells were ready for use. PMA was used to differentiate THP-1s from their monocyte-like form to a macrophage-like cell. THP-1s are incubated in RPMI media containing PMA at a concentration of 5 ng/ml for 48 hours.

Once ready, macrophages were then incubated with NC at a concentration of 50 uM overnight. The following day they were treated with P(I:C) at 50 ug/mL, 100 ug/mL, or 200 ug/mL, with LPS at 1 ng/ml or 10 ng/ml, or FSL at a concentration of 20 ng/ml or 50 ng/mL. Samples were treated for 4 hours and then Supernatant was then collected and the concentration of TNFα was then determined via the commercially available ELISA MAX Deluxe kit (Biolegend), with measurements reported in pg/mL.

### Results:

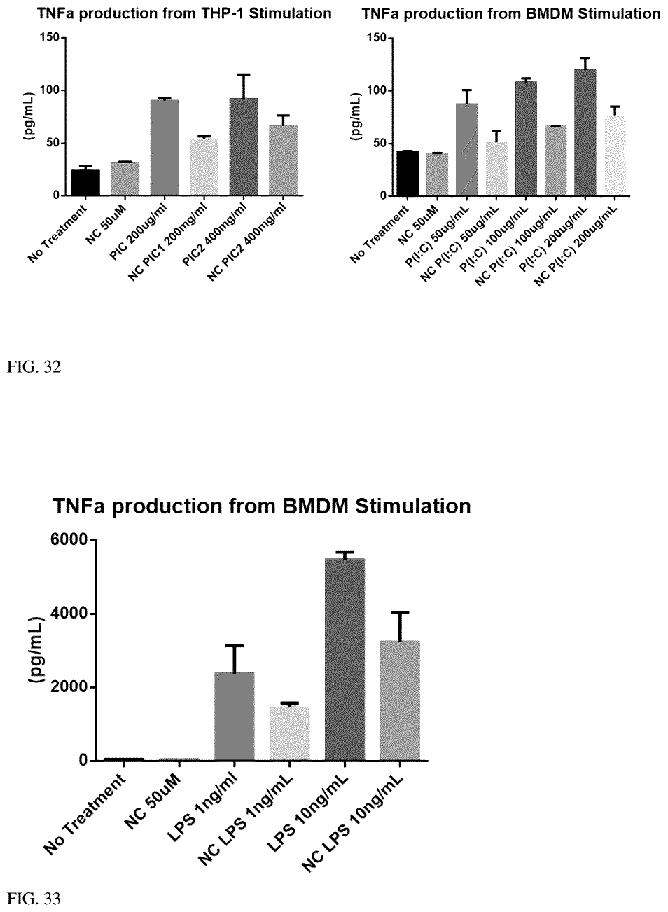

To investigate the effects NC, macrophages were stimulated in vitro with various TLR agonists, and the production of cytokines was measured via ELISA. Human THP-1 cells and primary BMDMs were seeded at a density of 2×10⁵ cells/well and were treated overnight with NC at a concentration of 50 μM and the following day they were stimulated with Poly (I:C) at a concentration of 50 μg/ml, 100 μg/ml, and 200 μg/ml for BMDMs and 200 mg/ml or 400 mg/mL for THP-1s. After 4 hours, supernatant was collected and the concentration of TNFα was determined via ELISA. NC alone was not found to increase production of TNFα, whereas pre-treatment with NC reduced the production and secretion of TNFα in both THP-1s and BMDMs (FIG. 32).

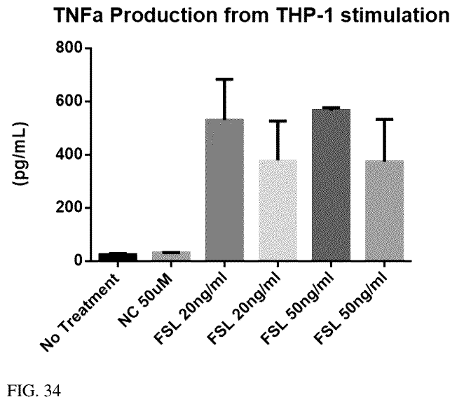

Further investigation of NC's impact on TLR signaling involved treating BMDMs with the TLR 4 agonist LPS. Following overnight treatment with NC cells were treated with LPS at a concentration of 1 ng/ml and 10 ng/ml. After 4 hours supernatant was collected and concentration of TNFα was measured via ELISA. NC was able to reduce cytokine production in response to TLR 4 agonist LPS (FIG. 33). Additionally, we explored NCs effects on other TLR agonists. Similarly to TLR 3 and 4, NC exhibited inhibitory effects to TLR 2/6 agonist FSL-1 (FIG. 34). We investigated NCs effects on various other TLR pathways including: ½ (PAM3CSK4), TLR 5 (Flagellin), TLR 7 (Imiquimod), and TLR 9 (ODN2006). Following the same pretreatment and stimulation protocol, and while testing various concentrations, NC did not exhibit any significant inhibitory effects on cytokine production.

1. A composition comprising
(a) one or more isolated bacterial metabolites selected from prednicarbate, N-palmitoyl-D-threonine and 3,3-difluoro-5-alpha-androstan-17-beta-yl-acetate, and
(b) a pharmaceutically acceptable carrier.

2. The composition of claim 1, comprising N-palmitoyl-D-threonine and a pharmaceutically acceptable carrier.

3. The composition of claim 1, comprising 3,3-difluoro-5-alpha-androstan-17-beta-yl-acetate and a pharmaceutically acceptable carrier.

4. The composition of claim 1, comprising prednicarbate, N-palmitoyl-D-threonine and a pharmaceutically acceptably carrier.

5. The composition of claim 1, comprising prednicarbate, 3,3-difluoro-5-alpha-androstan-17-beta-yl-acetate, and a pharmaceutically acceptable carrier.

6. The composition of claim 1, comprising prednicarbate, N-palmitoyl-D-threonine and 3,3-difluoro-5-alpha-androstan-17-beta-yl-acetate, and a pharmaceutically acceptable carrier.

7. The composition of claim 1, that is formulated for oral administration.

8. The composition of claim 1, further comprising one or more additional Lactobacillus metabolites (e.g., other than prednicarbate, N-palmitoyl-D-threonine, and 3,3-difluoro-5-alpha-androstan-17-beta-yl-acetate).

9. The composition of claim 8, wherein the one or more additional Lactobacillus metabolites have a molecular weight of less than 10 kD and are non-protein/DNA/RNA compounds (e.g., isolated from a low-MW supernatant fraction in a bacterial metabolite culture).

10. The composition of claim 1, wherein the composition is free from (i) compounds having a molecular weight of greater than 10 kD and (ii) protein/DNA/RNA compounds.

11. The composition of claim 1, wherein the composition is free from one or more of short-chain fatty acids, polyunsaturated fatty acids, and exopolysaccharides.

12. A method of reducing proinflammatory cytokine expression in a subject in need thereof, comprising administering the composition of claim 1 to the subject in an amount effective to reduce proinflammatory cytokine expression in the subject.

13. The method of claim 12, wherein the proinflammatory cytokine is IL6, TNFα, or IL1β.

14. A method of reducing inflammation in a subject in need thereof, comprising administering the composition of claim 1 to the subject in an amount effective to reduce inflammation.

15. A method of treating an inflammatory disorder in a subject in need thereof, comprising administering the composition of claim 1 to the subject in an amount effective to treat the disorder.

16. The method of claim 15, wherein the inflammatory disorder is dry eye disease.

17. The method of claim 15, wherein the inflammatory disorder is peritonitis.

18. The method of claim 15, wherein the inflammatory disorder is sepsis.
